(19) United States
(12) Patent Application Publication
(10) Pub. No.: US 2026/0198960 A1
(43) Pub. Date: Jul. 16, 2026

(54) MEDICAL DEVICE

(71) Applicant: TERUMO KABUSHIKI KAISHA, Tokyo (JP)

(72) Inventor: Kosuke NISHIO, Irvine, CA (US)

(73) Assignee: TERUMO KABUSHIKI KAISHA, Tokyo (JP)

(21) Appl. No.: 19/566,135

(22) Filed: Mar. 13, 2026

Related U.S. Application Data
(63) Continuation of application No. PCT/JP2024/023228, filed on Jun. 26, 2024.

(30) Foreign Application Priority Data
Sep. 29, 2023 (JP) 2023-169512

(51) Int. Cl. A61B 17/3207; A61B 17/22

(52) U.S. Cl. CPC A61B 17/320758; A61B 2017/22038

(57) ABSTRACT

A medical device that can increase operability and includes a cutter portion that can be rotated by a drive shaft is provided. A medical device that removes an object in a body lumen and includes: a rotary shaft including a rotatable hollow drive shaft and a hollow cutter portion to be rotatably driven by the drive shaft, a guide wire protecting tube disposed inside the drive shaft and the cutter portion, and a handle that rotatably houses the proximal portion of the rotary shaft. The guide wire protecting tube includes a small-diameter portion that forms the distal end of the guide wire protecting tube, and at least part of which is disposed inside the cutter portion, and the main body tube that is located on the proximal side with respect to the small-diameter portion, and at least part of which is disposed inside the drive shaft.

### CROSS-REFERENCES TO RELATED APPLICATIONS

This application is a continuation of International Application No. PCT/JP2024/023228 filed on Jun. 26, 2024, which claims priority to Japanese Application No. 2023-169512 filed on Sep. 29, 2023, the entire content of both of which is incorporated herein by reference.

### TECHNOLOGICAL FIELD

The present disclosure relates to a medical device for removing an object in a body lumen.

### BACKGROUND DISCUSSION

Treatment methods for treating a stenosed part due to a plaque, a thrombus, or the like in a blood vessel include a method for expanding the blood vessel with a balloon and a method for placing a meshed or coiled stent in the blood vessel as a support of the blood vessel, for example. However, with these methods, it is difficult to treat a stenosed part hardened due to calcification or a stenosed part formed in a bifurcation of a blood vessel. As a device that can provide treatment even in such a case, an atherectomy device that cuts a lesion and removes the cut object by suction has been proposed (see U.S. Patent Application Publication No. 2021/0275210 A, for example).

An atherectomy device includes a cutter portion that rotates at a high speed at the distal portion of the device, and a drive shaft that makes the cutter portion rotate. When an atherectomy device is used, it is necessary to insert a guide wire through the inside of the high-speed rotating cutter portion and the drive shaft, and there is a possibility that the guide wire will be damaged by friction with the inner surface of the cutter portion. Therefore, U.S. Patent Application Publication No. 2021/0275210 A proposes a structure in which a flexible protecting tube through which a guide wire can be inserted is disposed inside a cutter portion and a drive shaft, to protect the guide wire.

The protecting tube is inserted through the cutter portion and is disposed to reach the most distal end of the atherectomy device. Because of this, there is a possibility that the protecting tube protruding toward the distal side with respect to the cutter portion will degrade the passing properties of the atherectomy device for the object to be cut. To improve the passing properties of the atherectomy device, the protecting tube can be made thinner. However, if the protecting tube is made thinner, friction between the inner surface of the protecting tube and the outer surface of the guide wire would increase, and the operability of the atherectomy device would decrease.

### SUMMARY

A medical device is disclosed that can increase operability and includes a cutter portion that can be rotated by a drive shaft.
(1) A medical device according to the present disclosure is a medical device that removes an object in a body lumen, and includes: a rotary shaft including a drive shaft that is rotatable and hollow, and a cutter portion that is hollow and is rotatably driven by the drive shaft; a guide wire protecting tube disposed inside the rotary shaft; and a handle that rotatably houses a proximal portion of the rotary shaft, wherein the guide wire protecting tube includes: a small-diameter portion that forms a distal end of the guide wire protecting tube, and at least part of which is disposed inside the cutter portion; and a main body tube that is located on a proximal side with respect to the small-diameter portion, and at least part of which is disposed inside the drive shaft, and an inner diameter of the small-diameter portion is smaller than an inner diameter of the main body tube.

In the medical device disclosed above in (1), the inner diameter of the main body tube is larger than the inner diameter of the small-diameter portion, and accordingly, the contact resistance between the inner surface of the guide wire protecting tube and the outer surface of a guide wire can be reduced. Thus, the medical device can increase the operability of the guide wire extending through the inside of the guide wire protecting tube.
(2) In the medical device disclosed above in (1), an outer diameter of the small-diameter portion may be smaller than an outer diameter of the main body tube. Thus, the medical device can improve the passing properties of its distal area to pass through a stenosed part.
(3) In the medical device disclosed above in (1) or (2), a material forming the small-diameter portion may have a higher wear resistance than a material forming the main body tube. Thus, the medical device can reduce wear of the small-diameter portion that has a small inner diameter and easily comes into contact with the guide wire and prevent flow of wear debris into the body.
(4) In the medical device disclosed above in any one of (1) to (3), the cutter portion may include: a first housing portion having an inner diameter that allows housing of at least part of the small-diameter portion; a second housing portion having an inner diameter larger than the inner diameter of the first housing portion on the proximal side with respect to the first housing portion; and a first stepped portion that is located between the first housing portion and the second housing portion, and has a first stepped surface facing in a proximal direction, the guide wire protecting tube may include a first contact surface facing in a distal direction on the proximal side with respect to the small-diameter portion, an outer diameter of the small-diameter portion may be smaller than the inner diameter of the first housing portion, and the largest outer diameter of the first contact surface may be larger than the inner diameter of the first housing portion. Because of this, when the guide wire protecting tube moves in the distal direction more than necessary, the first contact surface of the guide wire protecting tube comes into contact with the first stepped surface of the cutter portion. Thus, the medical device can prevent the distal end of the guide wire protecting tube from protruding more than necessary from the distal end of the cutter portion. If the distal end of the guide wire protecting tube protrudes more than necessary from the distal end of the cutter portion, the cutter portion cannot come into contact with a stenosed part, and it is difficult to cut the stenosed part in some cases. However, the distal end does not protrude more than necessary, and thus, the stenosed part can be effectively cut by the cutter portion.
(5) In the medical device disclosed above in (4), the rotary shaft may include a third housing portion that is located on the proximal side with respect to the second housing portion, and has an inner diameter smaller than the inner diameter of the second housing portion, the guide wire protecting tube may have a second contact surface facing in the proximal direction on the proximal side with respect to the first contact surface, and the largest outer diameter of the second contact surface may be larger than the inner diameter of the third housing portion. Because of this, when the guide wire protecting tube moves in the proximal direction more than necessary, the second contact surface of the guide wire protecting tube comes into contact with the surface on the distal side of the third housing portion of the rotary shaft. Thus, the medical device can prevent the distal end of the guide wire protecting tube from moving to the proximal side with respect to the distal end of the cutter portion and prevent the guide wire extending through the guide wire protecting tube from coming into contact with the cutter portion.
(6) In the medical device disclosed above in any one of (1) to (5), the member including the small-diameter portion and the member including the main body tube may be separate members. Thus, the shape of the member including the small-diameter portion can be easily formed into a desired shape.
(7) In the medical device disclosed above in (5), a length in an axial direction between the first contact surface and the second contact surface of the guide wire protecting tube may be smaller than a length in the axial direction between the first stepped surface and the second stepped surface of the rotary shaft. Because of this, the guide wire protecting tube can move in the axial direction within a predetermined range inside the rotary shaft. Thus, it is possible to prevent the guide wire protecting tube from getting stuck in the axial direction inside the rotary shaft and hindering the rotation of the rotary shaft.
(8) In the medical device disclosed above in (4) or (5), the guide wire protecting tube may be fixed to a tube fixing portion of the handle, and a length from the tube fixing portion along the axis of the guide wire protecting tube to the first contact surface may be greater than a length from the tube fixing portion along the axis of the rotary shaft to the first stepped surface. Because of this, in a case where the rotary shaft is linear, the first contact surface of the guide wire protecting tube comes into contact with the first stepped surface of the rotary shaft, and the guide wire protecting tube is curved inside the rotary shaft. Further, in the state where the rotary shaft is bent after the medical device is inserted into the body, the amount of elongation in the axial direction of the guide wire protecting tube having a diameter smaller than that of the rotary shaft is smaller than the amount of elongation of the rotary shaft in the axial direction. That is, in a general case where a tubular body is curved, the amount of compression in the axial direction on the side located on the inner side of the curve and on which a compressive force acts is smaller than the amount of extension in the axial direction on the side located on the outer side of the curve and on which a tensile force acts. Therefore, the tubular body extends as a whole. The total amount of extension of the tubular body is larger in a case where the diameter of the tubular body is larger. Therefore, in a case where the medical device is inserted into a meandering lumen, the first contact surface of the guide wire protecting tube that has a smaller diameter than that of the rotary shaft and is less likely to extend because it is located inside the rotary shaft can be disposed at an appropriate distance from the first stepped surface. Thus, in an actual use state of the medical device being used in a meandering lumen, it is possible to prevent the guide wire protecting tube from getting stuck in the axial direction inside the rotary shaft and hindering the rotation of the rotary shaft.
(9) A medical device according to another embodiment includes: a rotary shaft including a drive shaft configured to be rotated and a cutting member that is configured to be rotatably driven by the drive shaft; and a guide wire protecting tube disposed inside the rotary shaft, the guide wire protecting tube includes a small-diameter portion, at least part of the small-diameter portion being disposed inside the cutting member, and a main body tube that is located on a proximal side with respect to the small-diameter portion, and at least part of the main body tube being disposed inside the drive shaft, and an inner diameter of the small-diameter portion is smaller than an inner diameter of the main body tube.
(10) A method for removing an object in a body lumen according to a further embodiment, includes: inserting a guide wire into a blood vessel and bringing and the guide wire into a vicinity of the object to be removed; inserting a proximal end of the guide wire lumen into a guide wire lumen of a medical device, the medical device including a rotary shaft including a drive shaft that is rotatable and hollow, and a cutter portion that is hollow and is rotatably driven by the drive shaft, a guide wire protecting tube disposed inside the rotary shaft, and a handle that rotatably houses a proximal portion of the rotary shaft, wherein the guide wire protecting tube includes a small-diameter portion, at least part of the small-diameter portion being disposed inside the cutter portion, and a main body tube that is located on a proximal side with respect to the small-diameter portion, and at least part of the main body tube being disposed inside the drive shaft, and an inner diameter of the small-diameter portion is smaller than an inner diameter of the main body tube; moving the cutter portion of the medical device to the vicinity of the object to be removed using the guide wire as a guide; and rotating the drive shaft and cutter portion to cut the object to be removed.

### BRIEF DESCRIPTION OF THE DRAWINGS

FIG. 1 is a plan view illustrating a medical device according to an embodiment.

FIG. 2 is a cross-sectional view illustrating a distal portion of the medical device.

FIG. 3 is a view illustrating the casing of the proximal portion of the medical device in a cross-sectional view, and the other portions in a plan view.

FIG. 4 is a cross-sectional view illustrating the vicinities of the distal portions of an inside tube and an outside tube of the medical device.

FIG. 5 is a schematic plan view illustrating a state of a guide wire protecting tube inside the medical device.

FIG. 6 is a schematic view illustrating a state in which the medical device is removing a lesion.

### DETAILED DESCRIPTION

Set forth below with reference to the accompanying drawings is a detailed description of embodiments of a medical device. Note that the size and ratio of each member in the drawings may be exaggerated for explanatory convenience and may be different from the actual size and ratio. Also, in the present specification, a side of a medical device to be inserted into a living body is referred to as the “distal side”, and a side to be operated is referred to as the “proximal side”.

A medical device 10 according to the present embodiment is inserted into a blood vessel in acute lower limb ischemia or deep-vein thrombosis and can be used for a treatment including destroying and removing an object such as a thrombus, a plaque, an atheroma, and a calcified lesion. Note that the object to be removed is not necessarily a thrombus, a plaque, an atheroma, and a calcified lesion, and may be any object that can be present in a body lumen.

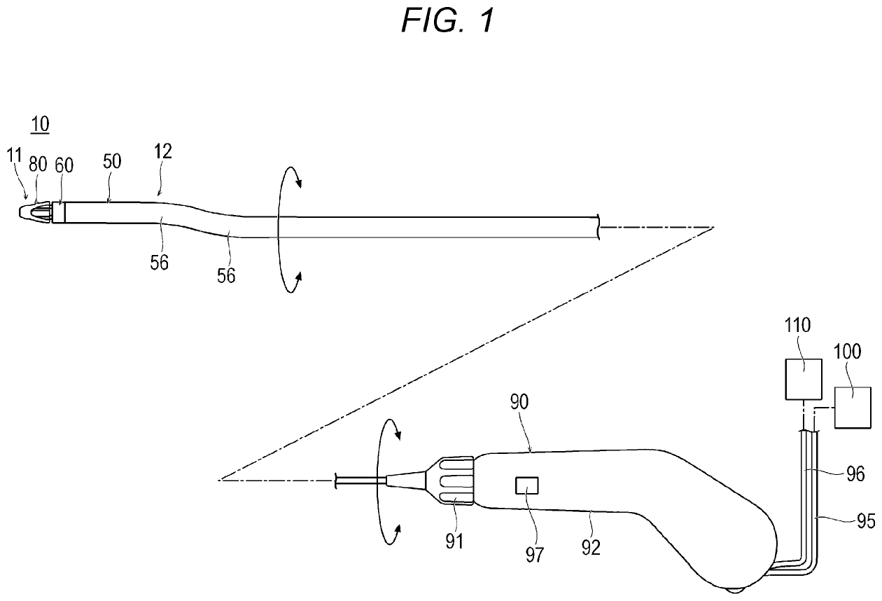

As illustrated in FIGS. 1 to 3, the medical device 10 includes a rotatable long rotary shaft 11, a housing shaft 12 that rotatably houses the rotary shaft 11, a guide wire protecting tube 40 disposed inside the rotary shaft 11, and a handle 90.

The rotary shaft 11 includes an elongated drive shaft 20, a conveyor coil 30 that conveys an object, for example, a thrombus, or a liquid, a cutter portion 80 that cuts an object such as a thrombus, and a connecting shaft 70 that connects the drive shaft 20 to the cutter portion 80.

The drive shaft 20 can be a multilayer coil in which coils are layered. The drive shaft 20 is flexible and has a property of being capable of transmitting a drive torque applied from the proximal side to the distal side. The drive shaft 20 is rotatable inside the housing shaft 12. The drive shaft 20 includes an inside coil 21, an outside coil 22 surrounding the outside of the inside coil 21, and a distal protecting tube 23 that covers the outer peripheral surface of the distal area of the outside coil 22. Note that the drive shaft 20 may be a multilayer coil including three or more layers. Further, the drive shaft 20 may be a single-layer coil.

The connecting shaft 70 is rotatably supported by a distal bearing portion 60, which will be described later, of the housing shaft 12. The proximal portion of the connecting shaft 70 is connected to the distal portion of the drive shaft 20, and the distal portion of the connecting shaft 70 enters the cutter portion 80 and is connected to the cutter portion 80. In the connecting shaft 70, at least one groove-like passage 71 extending along the axis is formed in the connecting shaft 70. The at least one groove-like passage 71 in the connecting shaft 70 allows an object cut by the cutter portion 80 to pass in a proximal direction through the inside of the distal bearing portion 60. In the connecting shaft 70, the guide wire protecting tube 40 is disposed in such a manner as to project through the connecting shaft 70. A third housing portion 73 is formed at the distal portion of the connecting shaft 70 that has entered the inside of a fitting portion 81 and is connected to the cutter portion 80, and a distal surface of the third housing portion 73 has a second stepped surface 72 facing in a distal direction.

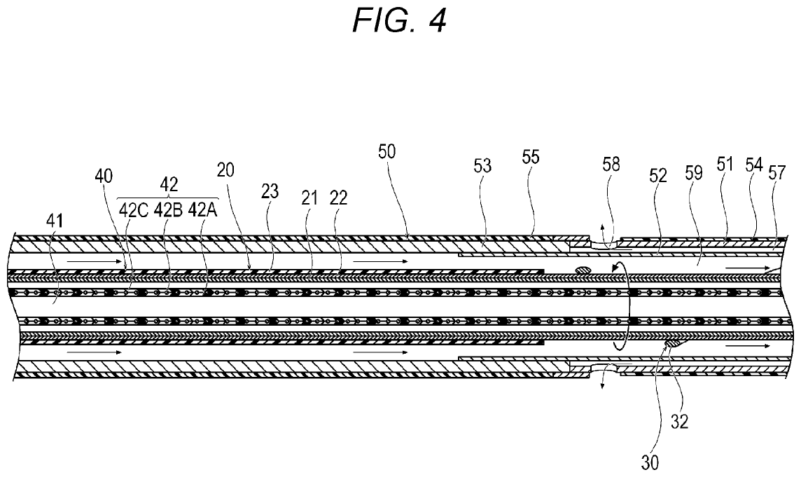

As illustrated in FIG. 4, the conveyor coil 30 is disposed so as to wrap around the outer periphery of the drive shaft 20. The distal portion of the conveyor coil 30 is fixed to the outer peripheral surface of the drive shaft 20. The conveyor coil 30 is formed by loosely winding the wire rod constituting the conveyor coil 30 so as to have a gap in the direction of the axis of the conveyor coil 30.

The conveyor coil 30 can function as an Archimedean screw (a screw pump) when the drive shaft 20 rotates in a rated rotational direction and conveys a liquid or an object in the proximal direction. Because of this, the conveyor coil 30 is wound in the rated rotational direction as it becomes closer to the distal side when viewed from the proximal side. Note that the conveyor coil 30 may be wound in a direction opposite to the rated rotational direction as it becomes closer to the distal side when viewed from the proximal side. In that case, the conveyor coil 30 can function as an Archimedean screw (a screw pump) when the drive shaft 20 rotates in the rated rotational direction and can convey a liquid or an object in the distal direction. Note that the conveyor coil 30 is not necessarily provided.

Examples of materials that can be preferably used for the drive shaft 20 and the conveyor coil 30 can include stainless steel, Ta, Ti, Pt, Au, W, polyolefin such as polyethylene and polypropylene, polyamide, polyester such as polyethylene terephthalate, fluorine-based polymer such as ethylene-tetrafluoroethylene copolymer (ETFE), polyether ether ketone (PEEK), and polyimide.

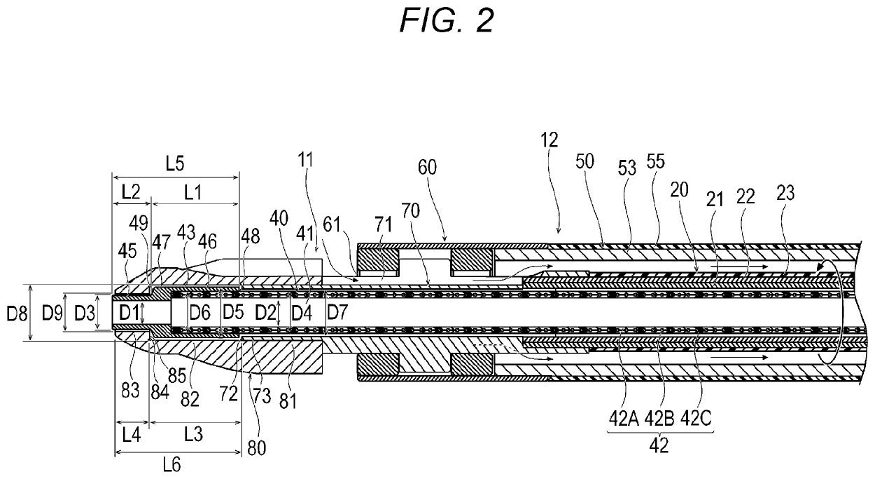

As illustrated in FIGS. 2 and 4, the distal protecting tube 23 is a tubular body that covers the outer peripheral surface of the drive shaft 20 on the distal side with respect to the conveyor coil 30. The distal protecting tube 23 is disposed inside a shaping portion 53 provided in an outer tube shaft 50. The distal protecting tube 23 is formed with a heat-shrinkable tube that is reduced in diameter by heating to be in close contact with the drive shaft 20, for example. The distal protecting tube 23 helps prevent the drive shaft 20 and the shaping portion 53 from coming into contact with each other and being damaged by the rotation of the drive shaft 20.

The material forming the heat-shrinkable tube is not limited to any particular one, and can be, for example, polyolefin, nylon, Pebax, polyurethane, or polyethylene terephthalate.

The cutter portion 80 is a member for cutting and shrinking an object such as a thrombus, a plaque, and a calcified lesion. Therefore, the term “cutting” is intended to mean an action of applying force to a contacting object and making the object smaller. The method of applying force during cutting, and the shape and the mode of the cut object are not limited to any particular ones. The cutter portion 80 is strong enough to cut the above-mentioned object. The cutter portion 80 is fixed to the outer peripheral surface of the distal portion of the connecting shaft 70. The cutter portion 80 can have a large number of fine abrasive grains on the surface of the cutter portion 80. Alternatively, the cutter portion 80 may have a sharp blade.

The cutter portion 80 has a through hole penetrating in the axial direction. The cutter portion 80 includes the fitting portion 81 located on the proximal side, a second housing portion 82 located on the distal side with respect to the fitting portion 81, a first housing portion 83 located on the distal side with respect to the second housing portion 82, and a first stepped portion 84 located between the second housing portion 82 and the first housing portion 83.

The fitting portion 81 is located at the proximal portion of the cutter portion 80, and the distal portion of the connecting shaft 70 enters the fitting portion 81 and fits in the fitting portion 81. Therefore, the inner diameter of the fitting portion 81 substantially coincides with the outer diameter of the distal portion of the connecting shaft 70. The second housing portion 82 is a portion formed continuously in the distal direction from the fitting portion 81. The inner diameter of the second housing portion 82 coincides with the inner diameter of the fitting portion 81 but does not necessarily coincide with the inner diameter of the fitting portion 81.

The first stepped portion 84 has a first stepped surface 85 facing in the proximal direction. The first stepped surface 85 is located at a distance from the second stepped surface 72 on the proximal side with respect to the second stepped surface 72 of the connecting shaft 70. The second housing portion 82 is disposed between the first stepped surface 85 and the second stepped surface 72.

The first housing portion 83 is located on the distal side with respect to the second housing portion 82 and has a smaller inner diameter than the inner diameter of the second housing portion 82. The first housing portion 83 forms an opening on the distal side of the cutter portion 80.

It is preferable that the material forming the cutter portion 80 is strong enough to cut a thrombus, and examples of the material of the cuter portion 80 that can be preferably used include stainless steel, Ta, Ti, Pt, Au, W, a shape memory alloy, and a supersteel alloy. The material forming the connecting shaft 70 preferably has a certain degree of strength, and the above-mentioned materials applicable to the cutter portion 80 can be used.

The guide wire protecting tube 40 is a tubular body disposed inside the rotary shaft 11, as illustrated in FIGS. 2 to 4. In the guide wire protecting tube 40, a guide wire lumen 41 through which a guide wire is inserted is formed. The guide wire protecting tube 40 is formed as a structure independent of the rotary shaft 11 and does not rotate with the rotary shaft 11. The guide wire protecting tube 40 helps prevent the guide wire extending through the guide wire lumen 41 from being rubbed against the drive shaft 20 as the rotary shaft 11, the connecting shaft 70, and/or the cutter portion 80. The guide wire protecting tube 40 includes a main body tube 42 forming most of the guide wire protecting tube 40, a distal member 43 fixed to the distal portion of the main body tube 42, and a proximal covering tube 44 fixed to the main body tube 42 so as to cover the proximal portion of the main body tube 42.

The main body tube 42 is a tubular body that is flexibly bendable so that its axis is bent. The main body tube 42 can be, for example, a braided tube in which a reinforcing member 42B is sandwiched between resin layers so as to have flexibility and strength. The main body tube 42 includes an inner layer 42A forming an inner surface, an outer layer 42C forming an outer surface, and the reinforcing member 42B located between the inner layer 42A and the outer layer 42C. The outer diameter of the main body tube 42 is smaller than the inner diameter of the connecting shaft 70 and is smaller than the inner diameter of the drive shaft 20.

The material forming the outer layer 42C is not limited to any particular material, and may be, for example, polyimide. The material forming the inner layer 42A is not limited to any particular material and may be a mixed material of polytetrafluoroethylene (PTFE) and polyimide, for example. Note that the inner layer 42A and the outer layer 42C may be an integrated structure formed with the same material.

The reinforcing member 42B is for reinforcing the main body tube 42 and is formed by braiding a plurality of wire rods in a tubular shape by a braider. The material of the outer layer 42C or the inner layer 42A enters a gap between the wire rods in the reinforcing member 42B. Note that the reinforcing member 42B is not necessarily provided. The material forming the reinforcing member 42B is not limited to any particular material, and may be, for example, a metal such as stainless steel, or a resin.

The distal member 43 includes a small-diameter portion 45 located at the distal portion of the distal member 43, a large-diameter portion 46 located at the proximal portion of the distal member 43, and a transition portion 47 located between the small-diameter portion 45 and the large-diameter portion 46.

The small-diameter portion 45 is rotatably housed in the first housing portion 83 of the cutter portion 80. An outer diameter D3 of the small-diameter portion 45 is smaller than an inner diameter D9 of the first housing portion 83. Because of this, the small-diameter portion 45 can rotate inside the first housing portion 83. Also, the outer diameter D3 of the small-diameter portion 45 is smaller than an outer diameter D4 of the main body tube 42. Because of this, the insertability of the small-diameter portion 45 protruding toward the distal side with respect to the cutter portion 80 into a stenosed part is increased. Note that the outer diameter D3 of the small-diameter portion 45 may be equal to the outer diameter D4 of the main body tube 42 or may be larger than the outer diameter D4. An inner diameter D1 of the small-diameter portion 45 is smaller than an inner diameter D2 of the main body tube 42. Because of this, the range (the small-diameter portion 45) in which the inner diameter of the guide wire protecting tube 40 is small and the frictional resistance with the guide wire is large is limited, and thus, the operability of the medical device 10 and the guide wire can be increased.

A length L2 of the small-diameter portion 45 in the axial direction is greater than a length L4 of the first housing portion 83 in the axial direction. Because of this, even if the guide wire protecting tube 40 moves in the axial direction inside the rotary shaft 11, the distal end of the small-diameter portion 45 can be positioned on the distal side of the distal end of the first housing portion 83, which is the distal end of the cutter portion 80. As a result, the guide wire housed in the guide wire protecting tube 40 can be prevented from being rubbed against the rotating cutter portion 80.

The large-diameter portion 46 is rotatably housed in the second housing portion 82 of the cutter portion 80. An outer diameter D5 of the large-diameter portion 46 is larger than the outer diameter D3 of the small-diameter portion 45 and is larger than the outer diameter D4 of the main body tube 42. The large-diameter portion 46 has a second contact surface 48 facing in the proximal direction on the proximal surface. The outer diameter D5 of the large-diameter portion 46 is smaller than an inner diameter D8 of the second housing portion 82. Because of this, the large-diameter portion 46 is rotatable inside the second housing portion 82. An inner diameter D6 of the large-diameter portion 46 substantially coincides with the outer diameter D4 of the main body tube 42 fitted to the large-diameter portion 46.

The transition portion 47 is located between the small-diameter portion 45 and the large-diameter portion 46. The inner diameter of the transition portion 47 coincides with the inner diameter D1 of the small-diameter portion 45. Note that the inner diameter of the transition portion 47 does not necessarily coincide with the inner diameter D1 of the small-diameter portion 45 and may be smaller at a portion closer to the distal end, as in a tapered shape, for example. The outer diameter of the proximal portion of the transition portion 47 coincides with the outer diameter D5 of the large-diameter portion 46. Further, the transition portion 47 has a first contact surface 49 facing in the distal direction on the outer peripheral surface. The smallest outer diameter of the first contact surface 49 coincides with the outer diameter D3 of the small-diameter portion 45, and the largest outer diameter of the first contact surface 49 coincides with the outer diameter D5 of the large-diameter portion 46. A length L1 in the axial direction between the first contact surface 49 and the second contact surface 48 is smaller than a length L3 in the axial direction between the first stepped surface 85 and the second stepped surface 72. Because of this, the distal member 43 is movable within a predetermined range (within the range of L3 to L1) in the axial direction inside the cutter portion 80.

The largest outer diameter of the first contact surface 49 is larger than the inner diameter D9 of the first housing portion 83. Because of this, the first contact surface 49 moving in the distal direction cannot pass through the inside of the first housing portion 83 and comes into contact with the first stepped surface 85.

The largest outer diameter of the second contact surface 48 is larger than an inner diameter D7 of the distal area of the connecting shaft 70. Because of this, the second contact surface 48 moving in the proximal direction cannot pass through the inside of the connecting shaft 70 and comes into contact with the second stepped surface 72.

In a state where the guide wire protecting tube 40 has moved in the proximal direction, and the second contact surface 48 is in contact with the second stepped surface 72, the distal end of the small-diameter portion 45 (the distal end of the guide wire protecting tube 40) is located on the distal side with respect to the distal end of the first housing portion 83 (the distal end of the cutter portion 80). That is, a length L5 in the axial direction from the second contact surface 48 to the distal end of the small-diameter portion 45 is greater than a length L6 in the axial direction from the second stepped surface 72 to the distal end of the first housing portion 83. Because of this, even in a state where the guide wire protecting tube 40 has moved in the proximal direction to the extent possible inside the rotary shaft 11, and the second contact surface 48 is in contact with the second stepped surface 72, the guide wire inserted into the guide wire protecting tube 40 can also be prevented from being rubbed against the cutter portion 80.

The distal member 43 is formed with a material having a higher wear resistance than the main body tube 42. The wear resistance can be evaluated by comparing wear rates, specific wear amounts, friction coefficients, or the like in a wear test under the same conditions. The distal member 43 is less likely to bend (has a higher flexural rigidity) and is less likely to be deformed than the main body tube 42. The material forming the distal member 43 is an engineering plastic, a ceramic, or a metal, for example. The engineering plastic may be a super engineering plastic. The engineering plastic (super engineering plastic) can be, for example, polyether ether ketone (PEEK), polyimide, polyetherimide, or polytetrafluoroethylene (PTFE).

The proximal covering tube 44 is a tubular body that is fixed to the outer peripheral surface of the proximal portion of the main body tube 42, to reinforce the proximal portion of the main body tube 42. The proximal covering tube 44 is a heat-shrinkable tube formed with polyethylene terephthalate, for example. The proximal covering tube 44 is fixed to a tube fixing portion 98 formed in a casing 92 of the handle 90. The tube fixing portion 98 is formed with an adhesive, for example. Thus, the guide wire protecting tube 40 can be prevented from rotating following the rotary shaft 11 on the outer side.

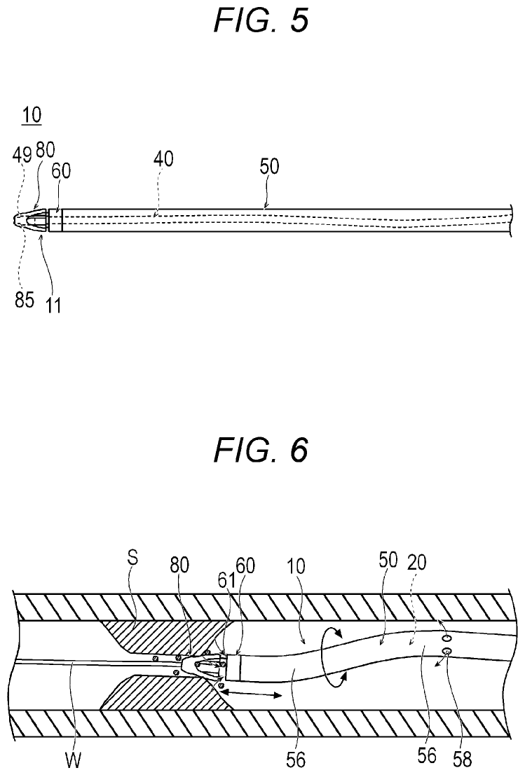

The length along the axis from the tube fixing portion 98 to the first contact surface 49 of the guide wire protecting tube 40 (the length in the axial direction from the tube fixing portion 98 to the first contact surface 49 in a case where the guide wire protecting tube 40 is linear without being curved) is greater than the length along the axis from the tube fixing portion 98 to the first stepped surface 85 of the rotary shaft 11 (the length in the axial direction from the tube fixing portion 98 to the first stepped surface 85 in a case where the rotary shaft 11 is linear without being curved). Therefore, in a case where the rotary shaft 11 is linear, as illustrated in FIG. 5, the first contact surface 49 of the guide wire protecting tube 40 comes into contact with the first stepped surface 85 of the rotary shaft 11, and the guide wire protecting tube 40 is curved inside the rotary shaft 11. In the state where the rotary shaft 11 is bent, the amount of elongation in the axial direction of the guide wire protecting tube 40 having a diameter smaller than that of the rotary shaft 11 is smaller than the amount of elongation of the rotary shaft 11 in the axial direction. That is, in a general case where a tubular body is curved, the amount of compression in the axial direction on the side located on the inner side of the curve and on which a compressive force acts is smaller than the amount of extension in the axial direction on the side located on the outer side of the curve and on which a tensile force acts. Therefore, the tubular body extends as a whole. The total amount of extension of the tubular body is larger in a case where the diameter of the tubular body is larger. Therefore, in a case where the medical device 10 is inserted into a meandering lumen, the first contact surface 49 of the guide wire protecting tube 40 that has a smaller diameter than that of the rotary shaft 11 and is less likely to extend because it is located inside the rotary shaft 11 can be disposed at an appropriate distance from the first stepped surface 85. Thus, in an actual use state of the medical device 10 being used in a meandering lumen, it is possible to prevent the guide wire protecting tube 40 from getting stuck in the axial direction inside the rotary shaft 11 and hindering the rotation of the rotary shaft 11.

As illustrated in FIGS. 1 to 4, the housing shaft 12 includes the outer tube shaft 50 that rotatably houses the rotary shaft 11, and the distal bearing portion 60 that rotatably supports the rotary shaft 11.

The outer tube shaft 50 includes an outside tube 51, an inside tube 52, the shaping portion 53, a first covering tube 54 in close contact with the outer peripheral surface of the outside tube 51, and a second covering tube 55 in close contact with the outer peripheral surface of the shaping portion 53. The outer tube shaft 50 is an elongated tubular body that houses the drive shaft 20. The outer tube shaft 50 can transmit a torque exerted by an operator on a rotating operation portion 91 fixed to the proximal portion of the outer tube shaft 50, to the distal side. A first lumen 57 for delivering a liquid such as a saline (or saline solution) to the distal side is formed between the outside tube 51 and the inside tube 52. At least one side hole 58 penetrating from the inner peripheral surface to the outer peripheral surface is formed at the distal portion of the outside tube 51. As the outer tube shaft 50 rotates, the cutter portion 80 can be made to face a lesion area.

It is preferable that the outside tube 51 has flexibility so as to bend in a body lumen and has a high torque transmissibility. Examples of the material that can be used for the outside tube 51 include materials obtained by forming a helical slit or groove by laser processing in a circular tube formed with a metal material or a resin material having a certain degree of strength. The material forming the outside tube 51 is not limited to any particular material, and examples of the material forming the outer tube 51 that can be preferably used include metal materials such as stainless steel, nitinol (NiTi), Ta, Ti, Pt, Au, and W, and engineering plastics such as ABS resin, polycarbonate (PC), polymethyl methacrylate (PMMA), polyacetal (POM), polyphenyl sulfone (PPSU), polyethylene (PE), carbon fiber, and polyether ether ketone (PEEK).

The first covering tube 54 is a tubular body that is in close contact with the outer peripheral surface of the outside tube 51. The first covering tube 54 reduces leakage of the liquid in the first lumen 57 from the gap of the helical slit formed in the outside tube 51. The first covering tube 54 is formed with a heat-shrinkable tube that is reduced in diameter by heating to be in close contact with the outside tube 51, for example.

The inside tube 52 is disposed inside the outside tube 51, with a gap being left in between the inside tube 52 and the outside tube 51. The gap between the inside tube 52 and the outside tube 51 is the first lumen 57. A second lumen 59 for discharging an object such as a cut thrombus in the proximal direction is formed inside the inside tube 52. The distal portion of the inside tube 52 is fixed to the inner peripheral surface of the shaping portion 53 with an adhesive or the like. The proximal portion of the inside tube 52 is located inside the handle 90.

The material forming the inside tube 52 desirably has a certain degree of flexibility and low frictional properties, and examples of the material forming the inside tube 52 that can be preferably used can include, for example, polyether ether ketone (PEEK), fluorine-based polymers such as PTFE and ETFE, polymethyl methacrylate (PMMA), polyethylene (PE), polyether block amide copolymer (PEBA) or PEBAX, nylon, polyimide, styrene-ethylene/butylene-styrene (SEBS), and combinations of the materials listed above. The inside tube 52 may have a braided reinforcement line.

The shaping portion 53 is located at the distal portion of the outer tube shaft 50. The shaping portion 53 is bent at two bent portions 56 so that the axis of the proximal portion of the shaping portion 53 and the axis of the distal portion deviate from each other. Note that the number of the bent portions 56 may be one or may be three or larger. By rotating the outer tube shaft 50, the shaping portion 53 can direct the cutter portion 80 toward the lesion area, and further strongly press the cutter portion 80 against the lesion area. As for the material forming the shaping portion 53, a material that can be used for the outside tube 51 described above can be adopted, for example.

The second covering tube 55 is a tubular body that is in close contact with the outer peripheral surface of the shaping portion 53. The second covering tube 55 is formed with a heat-shrinkable tube that is reduced in diameter by heating to be in close contact with the shaping portion 53, for example.

As illustrated in FIG. 2, the distal bearing portion 60 is fixed to the distal portion of the shaping portion 53 and is disposed to reach the distal side with respect to the shaping portion 53. The distal bearing portion 60 supports the connecting shaft 70 of the rotary shaft 11 in a rotatable manner. The distal bearing portion 60 has, on the distal side, a distal opening 61 through which an object such as a cut thrombus, blood, or a liquid discharged through the side hole 58 is conveyed and introduced into the second lumen 59. The distal end of the distal bearing portion 60 is located on the proximal side of the cutter portion 80. The material forming the distal bearing portion 60 preferably has a certain degree of strength, and any of the above-mentioned materials that can be used for the cutter portion 80 can be used.

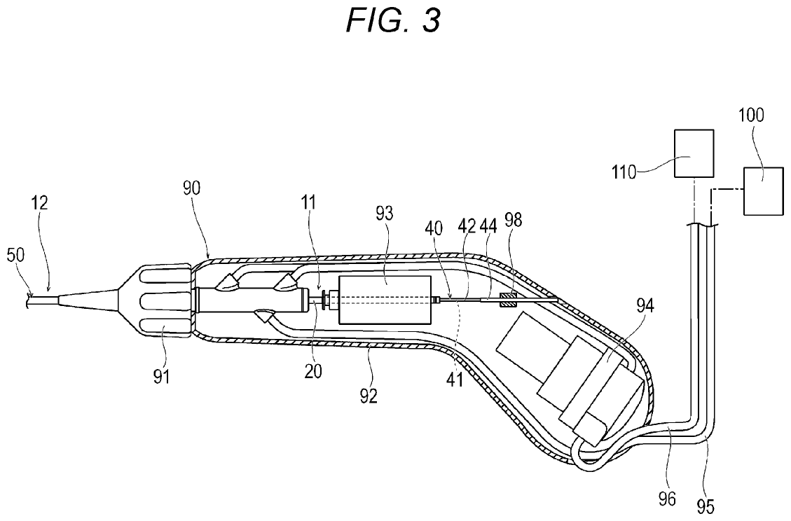

As illustrated in FIGS. 1 and 3, the handle 90 includes the rotating operation portion 91, the casing 92, a drive unit 93, a liquid feeder unit 94, a discharge port 95, a liquid feeding port 96, and an operation switch 97.

The casing 92 has the rotating operation portion 91 rotatably connected to the distal portion of the casing 92 and has the drive unit 93 and the liquid feeder unit 94 housed in the casing 92. Also, the proximal portion of the outer tube shaft 50 is disposed inside the casing 92.

The rotating operation portion 91 is a portion to be operated by an operator with their finger to apply a torque to the outer tube shaft 50. The rotating operation portion 91 is rotatably connected to the distal portion of the casing 92. The rotating operation portion 91 is fixed to the outer peripheral surface of the proximal portion of the outer tube shaft 50.

The drive unit 93 can be, for example, a hollow motor (i.e., electric motor with a hollow tubular structure). The drive unit 93 is rotated by a battery or power supplied from the outside. The drive shaft 20 is fixed to a hollow drive rotor of the hollow motor. The drive unit 93 is not particularly limited in rotation speed and may rotate at 10,000 revolutions per minute (rpm) to 150,000 rpm, or preferably 20,000 rpm to 120,000 rpm, for example. Note that the configuration of the drive unit 93 is not limited to any particular one. Since the drive unit 93 is a hollow motor, the guide wire protecting tube 40 and a guide wire can extend through the drive unit 93.

The discharge port 95 is a tube for discharging a liquid or an object to the outside of the casing 92. The discharge port 95 is connected to a waste liquid bag 100 that can store a liquid or an object, for example. Note that the discharge port 95 may be connected to a suction source that can actively suction, such as a pump or a syringe.

The liquid feeder unit 94 is a pump that feeds liquid to the first lumen 57. The liquid feeder unit 94 is connected to the liquid feeding port 96 that receives supply of a liquid such as saline (or saline solution) from a liquid supply 110 outside the casing 92 and can suction the liquid from the liquid feeding port 96 and discharge the liquid into the first lumen 57. The external liquid supply 110 is a saline bag, for example, but is not limited to this. The liquid feeder unit 94 may be provided outside, instead of being provided in the handle 90.

The operation switch 97 is a portion to be operated by an operator to activate or stop the drive unit 93. The operation switch 97 is located on the outer surface of the casing 92.

Next, a method of using the medical device 10 according to the embodiment is described. Here, a case where a calcified lesion area S in the blood vessel is broken up and conveyed as illustrated in FIG. 6 is described as an example.

First, the operator inserts a guide wire W into the blood vessel, and brings the guide wire W to the vicinity of the lesion area S. Next, the operator inserts the proximal end of the guide wire W into the guide wire lumen 41 of the medical device 10. After that, the operator moves the cutter portion 80 of the medical device 10 to the vicinity of the lesion area S, using the guide wire W as a guide.

Next, the operator operates the operation switch 97, to activate the drive unit 93 and the liquid feeder unit 94. Thus, the drive shaft 20 connected to the drive unit 93, and the connecting shaft 70 and the cutter portion 80 connected to the drive shaft 20 rotate. As a result, the operator can cut the lesion area S with the cutter portion 80. Further, as the drive shaft 20 rotates, the conveyor coil 30 fixed on the drive shaft 20 generates a force for conveying the liquid or the object in the second lumen 59 to the proximal side, as illustrated in FIG. 4. Thus, a suction force acts on the distal opening 61 of the second lumen 59 of the outer tube shaft 50, as illustrated in FIG. 2.

The operator can operate the rotating operation portion 91 illustrated in FIGS. 1 and 3, in a case where the position of the cutter portion 80 is to be changed in a circumferential direction. When the operator turns the rotating operation portion 91, the outer tube shaft 50 fixed to the rotating operation portion 91 rotates. As the outer tube shaft 50 rotates, the position and direction of the portion of the outer tube shaft 50 on the distal side with respect to the bent portions 56 change, and thus, the position and direction of the cutter portion 80 can be changed, as illustrated in FIG. 6. Accordingly, the operator can perform the cutting operation while changing the position and direction of the cutter portion 80 simply by operating the rotating operation portion 91, without rotating the entire handle 90 that is difficult to rotate greatly. Further, the operator moves the outer tube shaft 50 back and forth in the longitudinal direction of the blood vessel, by moving the entire handle 90 or the outer tube shaft 50 exposed to the outside of the body. Thus, the lesion area S can be cut in the longitudinal direction of the blood vessel with the cutter portion 80.

When the operation of the liquid feeder unit 94 is started, saline is drawn into the liquid feeder unit 94 through the liquid feeding port 96, and is discharged into the first lumen 57, as illustrated in FIGS. 1 to 4. The saline discharged into the first lumen 57 moves in the distal direction and is released into the blood vessel through the side hole 58 formed at the distal portion of the outside tube 51.

Part of the saline released into the blood vessel is drawn together with blood and the cut object into the second lumen 59 through the distal opening 61 of the outer tube shaft 50. The object has entered the second lumen 59 moves in the second lumen 59 in the proximal direction. The object that has entered the second lumen 59 is conveyed in the proximal direction by the conveyor coil 30 and is discharged into the external waste liquid bag 100 through the discharge port 95 of the handle 90.

Since the guide wire protecting tube 40 is fixed to the handle 90 by the tube fixing portion 98, it does not rotate together with the rotary shaft 11. Thus, the guide wire protecting tube 40 can prevent generation of wear debris due to rubbing of the guide wire W against the cutter portion 80.

After the cutting and conveyance of the lesion area S are completed, the operator operates the operation switch 97. Thus, the rotation of the drive shaft 20 is stopped, and the liquid feeding by the liquid feeder unit 94 is stopped. After that, the operator removes the medical device 10 from the blood vessel, and the treatment is completed.

As described above, the medical device 10 according to the present embodiment is the medical device 10 that removes an object in a body lumen, and includes: the rotary shaft 11 including the rotatable hollow drive shaft 20 and the hollow cutter portion 80 to be rotatably driven by the drive shaft 20; the guide wire protecting tube 40 disposed inside the rotary shaft 11; and the handle 90 that rotatably houses the proximal portion of the rotary shaft 11. The guide wire protecting tube 40 includes: the small-diameter portion 45 that forms the distal end of the guide wire protecting tube 40, and at least part of which is disposed inside the cutter portion 80; and the main body tube 42 that is located on the proximal side with respect to the small-diameter portion 45, and at least part of which is disposed inside the drive shaft 20. The inner diameter D1 of the small-diameter portion 45 is smaller than the inner diameter D2 of the main body tube 42. In the medical device 10, the inner diameter D2 of the main body tube 42 is larger than the inner diameter D1 of the small-diameter portion 45, and accordingly, the contact resistance between the inner surface of the guide wire protecting tube 40 and the outer surface of the guide wire W can be reduced. Thus, the medical device 10 can increase the operability of the guide wire W extending through the inside of the guide wire protecting tube 40.

The outer diameter D3 of the small-diameter portion 45 is smaller than the outer diameter D4 of the main body tube 42. Thus, the medical device 10 can improve the passing properties of its distal area to pass through a stenosed part.

The material forming the small-diameter portion 45 has a higher wear resistance than that of the material forming the main body tube 42. Thus, the medical device 10 can reduce wear of the small-diameter portion 45 that has a small inner diameter and easily comes into contact with the guide wire W and prevent flow of wear debris into the body.

The cutter portion 80 includes: the first housing portion 83 having the inner diameter D9 that allows housing of at least part of the small-diameter portion 45; the second housing portion 82 having the inner diameter D8 larger than the inner diameter D9 of the first housing portion 83 on the proximal side with respect to the first housing portion 83; and the first stepped portion 84 that is located between the first housing portion 83 and the second housing portion 82, and including the first stepped surface 85 facing in the proximal direction. The guide wire protecting tube 40 includes the first contact surface 49 facing in the distal direction on the proximal side with respect to the small-diameter portion 45. The outer diameter D3 of the small-diameter portion 45 is smaller than the inner diameter D9 of the first housing portion 83, and the largest outer diameter of the first contact surface 49 is larger than the inner diameter D9 of the first housing portion 83. Because of this, when the guide wire protecting tube 40 moves in the distal direction more than necessary, the first contact surface 49 of the guide wire protecting tube 40 comes into contact with the first stepped surface 85 of the cutter portion 80. Thus, the medical device 10 can help prevent the distal end of the guide wire protecting tube 40 from protruding more than necessary from the distal end of the cutter portion 80. If the distal end of the guide wire protecting tube 40 protrudes more than necessary from the distal end of the cutter portion 80, the cutter portion 80 cannot come into contact with a stenosed part, and it is difficult to cut the stenosed part in some cases. However, the distal end does not protrude more than necessary, and thus, the stenosed part can be effectively cut by the cutter portion 80.

The rotary shaft 11 includes the third housing portion 73 that is located on the proximal side with respect to the second housing portion 82 and has the inner diameter D7 smaller than the inner diameter D8 of the second housing portion 82. The guide wire protecting tube 40 includes the second contact surface 48 facing in the proximal direction on the proximal side with respect to the first contact surface 49, and the largest outer diameter of the second contact surface 48 is larger than the inner diameter D7 of the third housing portion 73. Because of this, when the guide wire protecting tube 40 moves in the proximal direction more than necessary, the second contact surface 48 of the guide wire protecting tube 40 comes into contact with the surface on the distal side of the third housing portion 73 of the rotary shaft 11. Thus, the medical device 10 can help prevent the distal end of the guide wire protecting tube 40 from moving to the proximal side with respect to the distal end of the cutter portion 80 and help prevent the guide wire W extending through the guide wire protecting tube 40 from coming into contact with the cutter portion 80.

The member including the small-diameter portion 45 and the member including the main body tube 42 are separate members. Thus, the shape of the member including the small-diameter portion 45 can be rather easily formed into a desired shape.

The length in the axial direction between the first contact surface 49 and the second contact surface 48 of the guide wire protecting tube 40 is smaller than the length in the axial direction between the first stepped surface 85 and the second stepped surface 72 of the rotary shaft 11. Because of this, the guide wire protecting tube 40 can move in the axial direction within a predetermined range inside the rotary shaft 11. Thus, it is possible to prevent the guide wire protecting tube 40 from getting stuck in the axial direction inside the rotary shaft 11 and hindering the rotation of the rotary shaft 11.

The guide wire protecting tube 40 is fixed to the tube fixing portion 98 of the handle 90, and the length from the tube fixing portion 98 to the first contact surface 49 along the axis of the guide wire protecting tube 40 is greater than the length from the tube fixing portion 98 to the first stepped surface 85 along the axis of the rotary shaft 11. Because of this, in a case where the rotary shaft 11 is linear, the first contact surface 49 of the guide wire protecting tube 40 comes into contact with the first stepped surface 85 of the rotary shaft 11, and the guide wire protecting tube 40 is curved inside the rotary shaft 11. Further, in the state where the rotary shaft 11 is bent after the medical device 10 is inserted into the body, the amount of elongation in the axial direction of the guide wire protecting tube 40 having a diameter smaller than that of the rotary shaft 11 is smaller than the amount of elongation of the rotary shaft 11 in the axial direction. That is, in a general case where a tubular body is curved, the amount of compression in the axial direction on the side located on the inner side of the curve and on which a compressive force acts is smaller than the amount of extension in the axial direction on the side located on the outer side of the curve and on which a tensile force acts. Therefore, the tubular body extends as a whole. The total amount of extension of the tubular body is larger in a case where the diameter of the tubular body is larger. Therefore, in a case where the medical device 10 is inserted into a meandering lumen, the first contact surface 49 of the guide wire protecting tube 40 that has a smaller diameter than that of the rotary shaft 11 and is less likely to extend because it is located inside the rotary shaft 11 can be disposed at an appropriate distance from the first stepped surface 85. Thus, in an actual use state of the medical device 10 being used in a meandering lumen, it is possible to help prevent the guide wire protecting tube 40 from getting stuck in the axial direction inside the rotary shaft 11 and hindering the rotation of the rotary shaft 11.

Note that the present disclosure is not limited to the embodiment described above, and those skilled in the art can make various modifications within the technical idea of the present invention. For example, the body lumen into which the medical device 10 is inserted is not limited to a blood vessel, and may be a vascular channel, a ureter, a bile duct, a fallopian tube, a hepatic duct, or the like, for example.

The detailed description above describes embodiments of a medical device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device that removes an object in a body lumen, the medical device comprising:
a rotary shaft including a drive shaft that is rotatable and hollow, and a cutter portion that is hollow and is rotatably driven by the drive shaft;
a guide wire protecting tube disposed inside the rotary shaft; and
a handle that rotatably houses a proximal portion of the rotary shaft, wherein
the guide wire protecting tube includes a small-diameter portion, at least part of the small-diameter portion being disposed inside the cutter portion, and a main body tube that is located on a proximal side with respect to the small-diameter portion, and at least part of the main body tube being disposed inside the drive shaft, and
an inner diameter of the small-diameter portion is smaller than an inner diameter of the main body tube.

2. The medical device according to claim 1, wherein an outer diameter of the small-diameter portion is smaller than an outer diameter of the main body tube.

3. The medical device according to claim 1, wherein a material forming the small-diameter portion has a higher wear resistance than a material forming the main body tube.

4. The medical device according to claim 1, wherein
the cutter portion includes:
a first housing portion having an inner diameter that allows housing of at least part of the small-diameter portion;
a second housing portion having an inner diameter larger than the inner diameter of the first housing portion on the proximal side with respect to the first housing portion; and
a first stepped portion that is located between the first housing portion and the second housing portion, and has a first stepped surface facing in a proximal direction;
the guide wire protecting tube includes a first contact surface facing in a distal direction on the proximal side with respect to the small-diameter portion; and
an outer diameter of the small-diameter portion is smaller than the inner diameter of the first housing portion, and a largest outer diameter of the first contact surface is larger than the inner diameter of the first housing portion.

5. The medical device according to claim 4, wherein
the guide wire protecting tube is fixed to a tube fixing portion of the handle; and
a length from the tube fixing portion along an axis of the guide wire protecting tube to the first contact surface is greater than a length from the tube fixing portion along an axis of the rotary shaft to the first stepped surface.

6. The medical device according to claim 4, wherein
the rotary shaft includes a third housing portion that is located on the proximal side with respect to the second housing portion, and has an inner diameter smaller than the inner diameter of the second housing portion; and
the guide wire protecting tube includes a second contact surface facing in the proximal direction on the proximal side with respect to the first contact surface, and
a largest outer diameter of the second contact surface is larger than the inner diameter of the third housing portion.

7. The medical device according to claim 6, wherein a length in an axial direction between the first contact surface and the second contact surface of the guide wire protecting tube is smaller than a length in the axial direction between the first stepped surface and the second stepped surface of the rotary shaft.

8. The medical device according to claim 1, wherein the small-diameter portion and the main body tube are separate members.

9. A medical device comprising:
a rotary shaft including a drive shaft configured to be rotated and a cutting member that is configured to be rotatably driven by the drive shaft; and
a guide wire protecting tube disposed inside the rotary shaft, the guide wire protecting tube includes a small-diameter portion, at least part of the small-diameter portion being disposed inside the cutting member, and a main body tube that is located on a proximal side with respect to the small-diameter portion, and at least part of the main body tube being disposed inside the drive shaft, and an inner diameter of the small-diameter portion is smaller than an inner diameter of the main body tube.

10. The medical device according to claim 9, wherein an outer diameter of the small-diameter portion is smaller than an outer diameter of the main body tube.

11. The medical device according to claim 9, wherein a material forming the small-diameter portion has a higher wear resistance than a material forming the main body tube.

12. The medical device according to claim 9, wherein the cutting member comprises:
a first housing portion having an inner diameter that allows housing of at least part of the small-diameter portion;
a second housing portion having an inner diameter larger than the inner diameter of the first housing portion on the proximal side with respect to the first housing portion; and
a first stepped portion, which is located between the first housing portion and the second housing portion, and the first stepped portion has a first stepped surface facing in a proximal direction.

13. The medical device according to claim 12, wherein the guide wire protecting tube includes a first contact surface facing in a distal direction on the proximal side with respect to the small-diameter portion, and an outer diameter of the small-diameter portion is smaller than the inner diameter of the first housing portion, and a largest outer diameter of the first contact surface is larger than the inner diameter of the first housing portion.

14. The medical device according to claim 13, wherein
the guide wire protecting tube is fixed to a tube fixing portion of a handle; and
a length from the tube fixing portion along an axis of the guide wire protecting tube to the first contact surface is greater than a length from the tube fixing portion along an axis of the rotary shaft to the first stepped surface.

15. The medical device according to claim 12, wherein
the rotary shaft includes a third housing portion that is located on the proximal side with respect to the second housing portion, and has an inner diameter smaller than the inner diameter of the second housing portion; and
the guide wire protecting tube includes a second contact surface facing in the proximal direction on the proximal side with respect to the first contact surface, and
a largest outer diameter of the second contact surface is larger than the inner diameter of the third housing portion.

16. The medical device according to claim 15, wherein a length in an axial direction between the first contact surface and the second contact surface of the guide wire protecting tube is smaller than a length in the axial direction between the first stepped surface and the second stepped surface of the rotary shaft.

17. A method for removing an object in a body lumen, the method comprising
inserting a guide wire into a blood vessel and bringing and the guide wire into a vicinity of the object to be removed;
inserting a proximal end of the guide wire lumen into a guide wire lumen of a medical device, the medical device including a rotary shaft including a drive shaft that is rotatable and hollow, and a cutter portion that is hollow and is rotatably driven by the drive shaft, a guide wire protecting tube disposed inside the rotary shaft, and a handle that rotatably houses a proximal portion of the rotary shaft, wherein the guide wire protecting tube includes a small-diameter portion, at least part of the small-diameter portion being disposed inside the cutter portion, and a main body tube that is located on a proximal side with respect to the small-diameter portion, and at least part of the main body tube being disposed inside the drive shaft, and an inner diameter of the small-diameter portion is smaller than an inner diameter of the main body tube;
moving the cutter portion of the medical device to the vicinity of the object to be removed using the guide wire as a guide; and
rotating the drive shaft and cutter portion to cut the object to be removed.

18. The method according to claim 17, further comprising:
conveying a liquid or the object within an outer tube of the medical device by the rotation of the shaft and a conveyor coil fixed on the drive shaft.

19. The method according to claim 17, wherein an outer diameter of the small-diameter portion is smaller than an outer diameter of the main body tube.

20. The method according to claim 17, wherein
the cutter portion includes:
a first housing portion having an inner diameter that allows housing of at least part of the small-diameter portion;
a second housing portion having an inner diameter larger than the inner diameter of the first housing portion on the proximal side with respect to the first housing portion; and
a first stepped portion that is located between the first housing portion and the second housing portion, and has a first stepped surface facing in a proximal direction;
the guide wire protecting tube includes a first contact surface facing in a distal direction on the proximal side with respect to the small-diameter portion; and
an outer diameter of the small-diameter portion is smaller than the inner diameter of the first housing portion, and a largest outer diameter of the first contact surface is larger than the inner diameter of the first housing portion.
